SEQUENCE LISTING (5 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 274 aa, Bacillus licheniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 3 - protein, 275 aa, Bacillus amyloliquefaciens [sequence omitted]

SEQ ID NO: 4 - protein, 269 aa, Bacillus gibsonii
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASLDGTSMATPHVAGVAALVKQKNPSWNATQIRNHLKNTATNLGNSSQFGSGLVNADAATR

SEQ ID NO: 5 - protein, 269 aa, Bacillus gibsonii
QQTVPWGITRVQAPAVHNRGITGSGVRVAILDSGISTHEDLNVRGGVSFVPGETTYADLNGHGTHVAGTVAALNNSIGVVGVAPSADLYAVKVLGANGRGSVSGIAQGLEWAAQNNMHIANMSLGAEGPSSTLERAVNYATSRDVLVIAATGNNGSGSVGYPARYANAMAVGATDQNNRRANFSQYGTGIDIVAPGVNVQSTYPGNRYVSMNGTSMATPHVAGAAALVKQRYPSWNATQIRDHLKNTATNLGNSSQFGSGLVNAEAATR

(19) United States
(12) Patent Application Publication
(10) Pub. No.: US 2026/0209735 A1
(43) Pub. Date: Jul. 23, 2026

(54) HIGH STRENGTH LIQUID PROTEASE FORMULATIONS

(71) Applicant: NOVOZYMES A/S, Bagsvaerd (DK)

(72) Inventor: Peter SORENSEN MILLARD, Albertslund (DK)

(73) Assignee: NOVOZYMES A/S, Bagsvaerd (DK)

(21) Appl. No.: 19/137,239

(22) PCT Filed: Dec. 14, 2023
(86) PCT No.: PCT/EP2023/085850

§ 371 Date: Jun. 9, 2025

(30) Foreign Application Priority Data
Dec. 14, 2022 (EP) 22213443.9

(51) Int. Cl. C12N 9/54; C11D 3/20; C11D 3/386; C12N 9/96

(52) U.S. Cl. CPC C12N 9/54; C11D 3/2065; C11D 3/386; C12N 9/96; C12Y 304/21062

(57) ABSTRACT

The invention provides high strength liquid protease compositions which are enzymatically and physically stable after storage.

### FIELD OF THE INVENTION

The present invention relates to high strength liquid protease formulations/compositions which are enzymatically and physically stable.

### BACKGROUND

Industrial enzymes are used in many different industries, such as household care, food, feed, and biofuels, and are supplied as both solid and liquid products. When liquid enzyme products are shipped across the world, and/or stored in warehouses, it is important that the products are sufficiently stable to maintain specifications, even when they reach the customers a long time after production. Stability includes both enzyme stability and physical stability.

The choice of formulation ingredients used to develop such stable formulations is not a simple one, because learnings from one enzyme class cannot be transferred to other enzyme classes. Further, liquid protease formulations are particularly difficult to make because the enzyme stability is challenged by autoproteolysis, in addition to compatibility issues with other formulation ingredients. Obviously, it also becomes increasingly difficult to keep the enzyme protein in solution, and retain a visually clear product, as the amount of enzyme protein gets higher.

### SUMMARY OF THE INVENTION

The present invention provides, in a first aspect, a liquid enzyme formulation, comprising
(a) 10-30% w/w active enzyme protein of protease,
(b) at least 40% w/w of glycerol, and
(c) less than 10% w/w of salt(s) of formate, acetate, citrate, chloride, or the corresponding acid(s); wherein pH of the formulation is in the range of 4-7.

Other aspects and embodiments of the invention are apparent from the description and examples.

Unless otherwise indicated, or if it is apparent from the context that something else is meant, all percentages are percentage by weight (% w/w).

As used herein, the term “consists essentially of” (and grammatical variants thereof), as applied to the compositions and methods of the invention, means that the compositions/methods may contain additional components so long as the additional components do not materially alter the composition/method.

As used herein, the term “essentially free of” (and grammatical variants thereof), as applied to the compositions and methods of the invention, means that the compositions/methods may contain minor amounts of the specified component so long as the amount of the component does not materially alter, or provide any material effect on, the composition/method. In an embodiment, “essentially free of” means 0% w/w.

### Sequences

SEQ ID NO: 1: amino acid sequence of a protease from Bacillus lentus.
SEQ ID NO: 2: amino acid sequence of a protease from Bacillus licheniformis.
SEQ ID NO: 3: amino acid sequence of a protease from Bacillus amyloliquefaciens.
SEQ ID NO: 4: amino acid sequence of a protease from Bacillus gibsonii.
SEQ ID NO: 5: amino acid sequence of a protease from Bacillus gibsonii.

### DETAILED DESCRIPTION

We have found that it is possible to make highly concentrated liquid protease formulations based on glycerol, which are visually clear and retains physical and enzymatic stability for several weeks at room temperature.

An advantage of such concentrated liquid formulations is that the amounts of solvent(s) and other formulation ingredients is low compared to the amount of protease. This results in lower material costs, less transportation, and less handling, which all reduces the environmental impact and carbon footprint of the products.

While most other protease formulations contain fossil-based polyols, like (mono) propylene glycol, the liquid formulation of the invention is based on glycerol, which is derived from plants and produced as a by-product of biofuel production.

Both physical and enzymatic stability is known to be difficult at high enzyme concentrations, and in particular at high protease concentrations because proteases can degrade themselves by autoproteolysis, resulting in reduced enzyme stability. (Mono) propylene glycol (MPG) is known to stabilize subtilisins and is used as the solvent of choice in most commercial liquid protease products. As shown by Joo et al., “Stabilization method of an alkaline protease from inactivation by heat, SDS and hydrogen peroxide”, Enzyme and Microbial Technology 36 (2005), propylene glycol provides superior stability compared to glycerol in certain liquid compositions. However, contrary to expectations, the inventors found that glycerol was a better protease solvent than propylene glycol at high protease concentrations (see Example 2).

Physical stability is the ability to maintain a transparent, preferably clear, composition. This may be evaluated by visual inspection, or by centrifugation. For example, the liquid composition may centrifugated at 1200 G for 10 minutes to determine if a pellet (solid phase) is formed. Alternatively, transparency may be measured as turbidity or haziness, by using a nephelometer to measure NTU to determine light scattering at 25° C. (see also U.S. EPA method 180.1).

Enzymatic stability is the ability to maintain enzymatic activity after storage. This may be determined by measuring the enzymatic activity before and after storage (for example, 4 weeks storage at 25° C.) to determine how much activity is lost. For practical purposes, the residual activity may be determined by comparing the activity of a stored sample and a frozen reference sample, which are analyzed at the same time to eliminate analytical day-to-day variation.

### Liquid Enzyme Composition

The liquid enzyme formulation of the invention comprises
(a) 10-30% w/w active enzyme protein of protease,
(b) at least 40% w/w of glycerol, and
(c) less than 10% w/w of salt(s) of formate, acetate, citrate, chloride, or the corresponding acid(s);
wherein pH of the formulation is in the range of 4-7.

The liquid formulation has excellent physical stability after storage (such as 4 weeks storage at 25° C.). In a preferred embodiment, the liquid formulation is visually transparent; or has essentially no solid phase after centrifugation at 1200 G for 10 minutes; or has a turbidity of less than 100 NTU, such as less than 50 NTU or less than 20 NTU, as measured using a nephelometer.

In an embodiment, the formulation is essentially free of benzoates, sorbates, sulfites, phenoxyethanol, and isothiozolinones (like methylisothiazolinone, chloromethylisothiazolinone, benzisothiazolinone, octylisothiazolinone, dichlorooctylisothiazolinone, and butylbenzisothiazolinone).

As mentioned above, the liquid formulation also maintains excellent enzymatic stability. The residual enzymatic activity may be at least 90% after 4 weeks storage at 25° C.

The liquid enzyme formulation may comprise more than 20% w/w (such as 20-50% w/w) of water; preferably more than 30% w/w (such as 30-50% w/w) of water, or more than 40% w/w (such as 40-50% w/w) of water. The water content depends on the other constituents of the formulation, as the total amount cannot exceed 100% (water ad 100% w/w).

Minor amounts of antioxidants or reducing agents like sulfite, thiosulfate, nitrite, ascorbic acid/ascorbate etc. may also be used to stabilize the protease (and the water phase in general). Other well-known stabilizers include divalent cations, like water-soluble magnesium and calcium salts.

### Protease

The proteases used in the liquid formulation of the invention are catalytic proteins, and the term “active enzyme protein” is defined herein as the amount of catalytic protein(s), which exhibits proteolytic activity. This can be determined using an activity based analytical enzyme assay. In such assays, the protease typically catalyzes a reaction generating a colored compound. The amount of the colored compound can be measured and correlated to the concentration of the active enzyme protein. This technique is well-known in the art.

The protease may be a serine protease, such as a subtilisin.

The protease may be a naturally occurring protease of bacterial or fungal origin, or it may be a variant derived from one or more naturally occurring proteases by gene shuffling and/or by substituting, deleting or inserting one or more amino acids. Chemically modified or protein engineered mutants are included.

A serine protease may for example be of the S1 family, such as trypsin, or the S8 family such as a subtilisin. A metalloprotease may for example be a thermolysin, e.g. from the M4 family, or another metalloprotease such as those from the M5, M7 or M8 families.

The term “subtilases” refers to a sub-group of serine proteases according to Siezen et al., Protein Eng. 4 (1991) 719-737 and Siezen et al., Protein Sci. 6 (1997) 501-523. Serine proteases are a subgroup of proteases characterized by having a serine in the active site, which forms a covalent adduct with the substrate. The subtilases may be divided into six subdivisions, the Subtilisin family, the Thermitase family, the Proteinase K family, the Lantibiotic peptidase family, the Kexin family and the Pyrolysin family.

Although proteases suitable for detergent use may be obtained from a variety of organisms, including fungi such as Aspergillus, detergent proteases have generally been obtained from bacteria and in particular from Bacillus. Examples of Bacillus species from which subtilases have been derived include Bacillus lentus, Bacillus alkalophilus, Bacillus subtilis, Bacillus amyloliquefaciens, Bacillus licheniformis, Bacillus pumilus and Bacillus gibsonii. Particular subtilisins include subtilisin lentus, subtilisin Novo, subtilisin Carlsberg, subtilisin BPN′, subtilisin 309, subtilisin 147 and subtilisin 168 and e.g. protease PD138 (described in WO 93/18140). Other useful proteases are e.g. those described in WO 01/16285 and WO 02/16547.

In an embodiment of the invention, the amino acid sequence of the subtilisin has at least 80% sequence identity, preferably at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1, SEQ ID NO: 2, SEQ ID NO: 3, SEQ ID NO: 4, or SEQ ID NO: 5.

Amino acid alterations, as described above, may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant molecules are tested for protease activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596:583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, CRISPR gene editing, phage display (e.g., Lowman et al., 1991, Biochemistry 30:10832-10837; U.S. Pat. No. 5,223,409; WO 92/06204), and region-directed mutagenesis (Derbyshire et al., 1986, Gene 46:145; Ner et al., 1988, DNA 7:127).

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

Suitable commercially available protease enzymes include those sold under the trade names Alcalase, Duralase, Durazym, Relase, Relase Ultra, Savinase, Savinase Ultra, Primase, Polarzyme, Kannase, Liquanase, Liquanase Ultra, Ovozyme, Coronase, Coronase Ultra, Blaze, Neutrase, Everlase, Esperase, Progress Uno, Progress Key and Progress Excel (Novozymes), those sold under the tradename Maxatase, Maxacal, Maxapem, Purafect, Purafect Prime, Purafect MA, Purafect Ox, Purafect OxP, Puramax, Properase, FN2, FN3, FN4, Excellase, Eraser, Opticlean, Optimase, Preferenz P200, and Preferenz P300 (DuPont/IFF), BLAP (sequence shown in FIG. 29 of U.S. Pat. No. 5,352,604) and variants hereof (Henkel AG), KAP (Bacillus alkalophilus subtilisin from Kao), and Lavergy Pro (BASF).

The liquid formulation of the invention comprises the protease (or subtilisin) in an amount of at least 10% w/w active enzyme protein, such as at least 11% w/w, at least 12% w/w, at least 13% w/w, at least 14% w/w, or at least 15% w/w active enzyme protein. The liquid formulation comprises at most 30% w/w active enzyme protein, such as at most 25% w/w active enzyme protein.

In an embodiment, the liquid formulation comprises less than 1% w/w of other (non-protease) enzyme(s); preferably is essentially free of other (non-protease) enzyme(s).

### Polyol

The liquid formulation comprises at least 40% w/w of glycerol, such as at least 45% w/w, at least 50% w/w, at least 55% w/w, or at least 60% w/w of glycerol.

In an embodiment, the liquid composition comprises less than 15% w/w, preferably less than 10% w/w, less than 5% w/w, or less than 2% w/w, of (mono) propylene glycol. The liquid composition may be essentially free of (mono) propylene glycol. Preferably, (mono) propylene glycol is 1,2-propylene glycol.

In another embodiment, the liquid composition comprises less than 15% w/w, preferably less than 10% w/w, less than 5% w/w, or less than 2% w/w, of other polyols than glycerol. The liquid composition may be essentially free of other polyols than glycerol.

Polyols (or polyhydric alcohols) according to the invention are alcohols with two or more hydroxyl groups. The polyols typically have a molecular weight lower than 500 g/mol.

Polyols include non-sugar polyols, such as glycerol, ethylene glycol, diethylene glycol, triethylene glycol, propylene glycol, dipropylene glycol, tripropylene glycol, polyethylene glycol (PEG), and sugar alcohols. The polyethylene glycol may have an average molecular weight at or below about 500. Examples of sugar alcohols are sorbitol, mannitol, erythritol, dulcitol, inositol, xylitol and adonitol.

Polyols also include sugar polyols, such as mono- and disaccharides, like glucose, fructose, galactose, sucrose, lactose, maltose, and trehalose.

### Salt

Salts and the corresponding acids are often used as buffers in liquid formulations. However, the liquid formulation of the invention comprises such high amounts of enzyme that side chains of the amino acid residues can provide the liquid formulation with buffering capacity without adding separate buffers.

The liquid formulation comprises less than 10% w/w, preferably less than 8% w/w, less than 6% w/w, less than 4% w/w, less than 2% w/w, or less than 1% w/w, of salt(s) of formate, acetate, citrate, chloride, or the corresponding acids. The amount of salt(s) is calculated as unhydrated salt, thus excluding any complexed water (crystal water). Preferred salts are sodium and potassium salts of formate, acetate, citrate, and chloride.

In an embodiment, the liquid formulation comprises less than 10% w/w, preferably less than 8% w/w, less than 6% w/w, less than 4% w/w, less than 2% w/w, or less than 1% w/w, of salt(s) of any kind or the corresponding acids.

### Protease Inhibitors

Proteases, as described above, may be stabilized using compounds that act by temporarily reducing the proteolytic activity (reversible inhibitors).

Thus, the composition of the invention may also include a protease inhibitor, which is a reversible inhibitor of protease activity. Preferably, the protease inhibitor is a (reversible) subtilisin protease inhibitor. In particular, the protease inhibitor may be a peptide aldehyde, boric acid, a boronic acid; or a derivative of any of these. Examples of protease inhibitors are shown in, for example, WO 96/041859, WO 2009/118375, WO 2010/055052, and WO 2013/004636.

In an embodiment, the protease inhibitor is phenyl-boronic acid or a derivative thereof, such as a formyl-phenyl-boronic acid (for example 2-FPBA, 3-FPBA, or 4-FPBA). In a particular embodiment, the protease inhibitor is 4-formyl-phenyl-boronic acid (4-FPBA).

Other examples of suitable boronic acids include thiophene-2 boronic acid, thiophene-3 boronic acid, acetamidophenyl boronic acid, benzofuran-2 boronic acid, naphtalene-1 boronic acid, naphtalene-2 boronic acid, 1-thianthrene boronic acid, 4-dibenzofuran boronic acid, 5-methylthiophene-2 boronic, acid, thionaphtrene boronic acid, furan-2 boronic acid, furan-3 boronic acid, 4,4 biphenyl-diborinic acid, 6-hydroxy-2-naphtalene, 4-(methylthio)phenyl boronic acid, 4 (trimethyl-silyl)phenyl boronic acid, 3-bromothiophene boronic acid, 4-methylthiophene boronic acid, 2-naphtyl boronic acid, 5-bromothiphene boronic acid, 5-chlorothiophene boronic acid, dimethylthiophene boronic acid, 2-bromophenyl boronic acid, 3-chlorophenyl boronic acid, 3-methoxy-2-thiophene, p-methyl-phenylethyl boronic acid, 2-thianthrene boronic acid, di-benzothiophene boronic acid, 4-carboxyphenyl boronic acid, 9-anthryl boronic acid, 3,5 dichlorophenyl boronic, acid, diphenyl boronic acidanhydride, o-chlorophenyl boronic acid, p-chlorophenyl boronic acid, m-bromophenyl boronic acid, p-bromophenyl boronic acid, p-flourophenyl boronic acid, p-tolyl boronic acid, o-tolyl boronic acid, octyl boronic acid, 1,3,5 trimethylphenyl boronic acid, 3-chloro-4-flourophenyl boronic acid, 3-aminophenyl boronic acid, 3,5-bis-(triflouromethyl) phenyl boronic acid, 2,4 dichlorophenyl boronic acid, and 4-methoxyphenyl boronic acid.

In another embodiment, the protease inhibitor is a peptide aldehyde having the formula P-B2-B1-B0-H, wherein
B0 is an amino acid selected from the group consisting of arginine (Arg), 3,4-dihydroxyphenylalanine, isoleucine (Ile), leucine (Leu), methionine (Met), norleucine (Nle), norvaline (Nva), phenylalanine (Phe), m-tyrosine, p-tyrosine (Tyr), and valine (Val);
B1 is an amino acid selected from the group consisting of alanine (Ala), cysteine (Cys), glycine (Gly), isoleucine (Ile), leucine (Leu), norleucine (Nle), norvaline (Nva), proline (Pro), serine (Ser), threonine (Thr), and valine (Val);
B2 is an amino acid selected from the group consisting of alanine (Ala), arginine (Arg), capreomycidine (Cpd), cysteine (Cys), glycine (Gly), isoleucine (Ile), leucine (Leu), norleucine (Nle), norvaline (Nva), phenylalanine (Phe), proline (Pro), serine (Ser), threonine (Thr), and valine (Val);
P is a N-terminal protection group, which may be selected from formyl, acetyl (Ac), benzoyl (Bz), trifluoroacetyl, methoxysuccinyl, aromatic and aliphatic urethane protecting groups such as fluorenylmethyloxycarbonyl (Fmoc), methoxycarbonyl (Moc), (fluoromethoxy) carbonyl, benzyloxycarbonyl (Cbz), t-butyloxycarbonyl (Boc) and adamantyloxycarbonyl; p-methoxybenzyl carbonyl, benzyl (Bn), p-methoxybenzyl (PMB), p-methoxyphenyl (PMP), methoxyacetyl, methylamino carbonyl, methylsulfonyl, ethylsulfonyl, benzylsulfonyl, methylphosphoramidyl (MeOP(OH)(═O)) and benzylphosphoramidyl (PhCH₂OP(OH)(═O)); and
H is hydrogen.

In a preferred embodiment,
B0 is leucine (Leu), methionine (Met), phenylalanine (Phe), tyrosine (Tyr), or valine (Val);
B1 is alanine (Ala), glycine (Gly), or valine (Val);
B2 is arginine (Arg), glycine (Gly), leucine (Leu), phenylalanine (Phe), or valine (Val);
P is benzyloxycarbonyl (Cbz) or methoxycarbonyl (Moc); and
H is hydrogen.

In a particular embodiment, the peptide aldehyde has the formula Cbz-Gly-Ala-Tyr-H or Cbz-Val-Ala-Leu-H.

### Production

The protease comprised in the liquid formulation of the invention is usually produced by a fermentation and subsequent recovery process. The fermentation liquid/broth may be subjected to a flocculation/precipitation step to provide a purified protease supernatant, and subsequently the purified protease supernatant may be subjected to a membrane filtration to provide a concentrated protease solution. Preferably, the membrane filtration comprises an ultra-filtration. The concentrated protease solution may subsequently be used to produce the liquid formulation of the invention in a process that comprises mixing the concentrated protease solution with glycerol, and optionally evaporating some water at a partial vacuum to increase the protease concentration. Water may also be evaporated from the concentrated protease solution before adding the glycerol.

Depending on the desired purity, the fermentation broth (from the fermentation), the protease supernatant (from the flocculation), or the concentrated protease solution (from the membrane filtration) may be subjected to spray-drying (or freeze drying) to provide a protease powder. The protease powder may subsequently be used to produce the liquid formulation of the invention in a process that comprises mixing the protease powder with water and glycerol.

### Recovery

In order to flocculate the fermentation broth a divalent salt may be added to the fermentation broth, in particular a calcium salt and/or a magnesium salt, e.g., calcium chloride or magnesium chloride. A preferred embodiment is a calcium salt, in particular calcium chloride.

Many aluminium compounds are known to improve flocculation, e.g., Al₂(SO₄)₃, NaAlO₂, K₂Al₂O₄, AlCl₃, Al(NO₃)₃, Al-acetate, and Al-formate. Particularly useful poly aluminium chlorides include compounds of the formula Alₙ(OH)ₘCl₍₃ₙ₋ₘ₎ and poly aluminium chlorides and aluminium chlorohydrates with the CAS No.: 1327-41-9.

The flocculation salt(s) may be added to the fermentation broth in a concentration of 0.01-10% (w/w) per kg fermentation broth (un-diluted); preferably 0.5-10% (w/w) per kg fermentation broth (un-diluted); more preferably 1-9% (w/w) per kg fermentation broth (un-diluted); in particular 2-8% (w/w) per kg fermentation broth (un-diluted).

Polymers may be used for particle aggregation. Anionic and cationic polymers are preferred. A useful cationic polymer may be a polyamine, and a useful anionic polymer may be a polyacrylamid. Useful polymer concentrations will normally be in the range of 0.5-20% (w/w) calculated per kg fermentation broth (un-diluted); preferably in the range of 1-10% (w/w) calculated per kg fermentation broth (un-diluted).

An example of a useful anionic polymer is Superfloc™ A 130 (Kemira). Examples of useful cationic polymers are Polycat™ (Kemira), C521 (Kemira), and C591 (Kemira).

The flocculated cell debris may be removed by methods known in the art such as, but not limited to, filtration, e.g., drum filtration, membrane filtration, filter-press dead end filtration, cross-flow filtration, or centrifugation.

The resulting fermentation supernatant may then be further processed or refined by methods known in the art. For example, the protein may be recovered by conventional procedures including, but not limited to, further filtration such as ultra-filtration and dia-filtration, extraction, spray-drying, evaporation, precipitation or crystallization.

### Uses

The liquid formulation of the invention may be used to produce enzymatic detergents, enzymatic feeds, foods, or be applied as a process catalyst in various industrial processes.

Thus, a liquid detergent composition may be produced by a process that comprises mixing the liquid enzyme formulation of the invention with a surfactant and a detergent builder. The final concentration of the protease in the detergent may be 0.0001-1% w/w active enzyme protein, preferably 0.0005-0.5% w/w active enzyme protein.

The surfactant may be anionic, such as linear alkylbenzenesulfonates (LAS), isomers of LAS such as branched alkylbenzenesulfonates (BABS) and phenylalkanesulfonates; olefin sulfonates, in particular alpha-olefinsulfonates (AOS); alkyl sulfates (AS), in particular fatty alcohol sulfates (FAS), i.e., primary alcohol sulfates (PAS) such as dodecyl sulfate; alcohol ethersulfates (AES or AEOS or FES, also known as alcohol ethoxysulfates or fatty alcohol ether sulfates); paraffin sulfonates (PS) including alkane-1-sulfonates and secondary alkanesulfonates (SAS); ester sulfonates, including sulfonated fatty acid glycerol esters and alpha-sulfo fatty acid methyl esters (alpha-SFMe or SES or MES); alkyl- or alkenylsuccinic acids such as dodecenyl/tetradecenyl succinic acid (DTSA); diesters and monoesters of sulfosuccinic acid; fatty acid derivatives of amino acids.

The surfactant may be non-ionic, such as alcohol ethoxylates (AE or AEO) e.g. the AEO-series such as AEO-7, alcohol propoxylates, in particular propoxylated fatty alcohols (PFA), ethoxylated and propoxylated alcohols, alkoxylated fatty acid alkyl esters, such as ethoxylated and/or propoxylated fatty acid alkyl esters (in particular methyl ester ethoxylates, MEE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA).

The surfactant may be a glycolipid selected from the group consisting of sophorolipid, rhamnolipid, trehalolipid, and mannosylerythritol lipid; or a lipopeptide, such as surfactin.

The final concentration of the surfactant may be 0.5-40% w/w, preferably 0.5-25% w/w.

The detergent builder may be citrate, aminocarboxylates, aminopolycarboxylates, phosphonates, alkyl- or alkenylsuccinic acid, 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N, N-diacetic acid (GLDA), 1-hydroxyethane-1,1-diylbis(phosphonic acid (HEDP), ethylenediaminetetramethylenetetrakis(phosphonic acid) (EDTMPA), diethylenetriaminepentamethylenepentakis (phosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), N-methyliminodiacetic acid (MIDA), serine-N,N-diacetic acid (SEDA), isoserine-N,N-diacetic acid (ISDA), phenylalanine-N, N-diacetic acid (PHDA), anthranilic acid-N,N-diacetic acid (ANDA), sulfanilic acid-N, N-diacetic acid (SLDA), taurine-N,N-diacetic acid (TUDA) and sulfomethyl-N, N-diacetic acid (SMDA), N-(2-hydroxyethyl)ethylenediamine-N,N′,N″-triacetic acid (HEDTA), diethanolglycine (DEG), aminotrimethylenetris(phosphonic acid) (ATMP), or salts thereof.

The final concentration of the detergent builder may be 0.5-40% w/w, preferably 0.5-25% w/w.

Further embodiments of the invention include:

Embodiment 1. A liquid enzyme formulation, comprising
(a) 10-30% w/w active enzyme protein of protease,
(b) at least 40% w/w of glycerol, and
(c) less than 10% w/w of salt(s) of formate, acetate, citrate, chloride, or the corresponding acid(s);
wherein pH of the formulation is in the range of 4-7.

Embodiment 2. The enzyme formulation of the preceding embodiment, wherein the protease is a serine protease.

Embodiment 3. The enzyme formulation of the preceding embodiment, wherein the protease is a subtilisin.

Embodiment 4. The enzyme formulation of any of the preceding embodiments, which comprises less than 15% w/w of (mono) propylene glycol, preferably (mono) propylene glycol is 1,2-propylene glycol.

Embodiment 5. The enzyme formulation of any of the preceding embodiments, which comprises less than 10% w/w of (mono) propylene glycol, preferably (mono) propylene glycol is 1,2-propylene glycol.

Embodiment 6. The enzyme formulation of any of the preceding embodiments, which comprises less than 5% w/w of (mono) propylene glycol, preferably (mono) propylene glycol is 1,2-propylene glycol.

Embodiment 7. The enzyme formulation of any of the preceding embodiments, which comprises less than 2% w/w of (mono) propylene glycol, preferably (mono) propylene glycol is 1,2-propylene glycol.

Embodiment 8. The enzyme formulation of any of the preceding embodiments, which is essentially free of (mono) propylene glycol, preferably (mono) propylene glycol is 1,2-propylene glycol.

Embodiment 9. The enzyme formulation of any of the preceding embodiments, which comprises less than 15% w/w of other polyols than glycerol.

Embodiment 10. The enzyme formulation of any of the preceding embodiments, which comprises less than 10% w/w of other polyols than glycerol.

Embodiment 11. The enzyme formulation of any of the preceding embodiments, which comprises less than 5% w/w of other polyols than glycerol.

Embodiment 12. The enzyme formulation of any of the preceding embodiments, which comprises less than 2% w/w of other polyols than glycerol.

Embodiment 13. The enzyme formulation of any of the preceding embodiments, which is essentially free of other polyols than glycerol.

Embodiment 14. The enzyme formulation of any of the preceding embodiments, which comprises at least 45% w/w of glycerol.

Embodiment 15. The enzyme formulation of any of the preceding embodiments, which comprises at least 50% w/w of glycerol.

Embodiment 16. The enzyme formulation of any of the preceding embodiments, which comprises at least 55% w/w of glycerol.

Embodiment 17. The enzyme formulation of any of the preceding embodiments, which comprises at least 60% w/w of glycerol.

Embodiment 18. The enzyme formulation of any of the preceding embodiments, which comprises less than 5% w/w of salt(s) of formate, acetate, citrate, chloride, or the corresponding acid(s).

Embodiment 19. The enzyme formulation of any of the preceding embodiments, which comprises less than 2% w/w of salt(s) of formate, acetate, citrate, chloride, or the corresponding acid(s).

Embodiment 20. The enzyme formulation of any of the preceding embodiments, which comprises less than 10% w/w of salt(s), or the corresponding acid(s).

Embodiment 21. The enzyme formulation of any of the preceding embodiments, which comprises less than 5% w/w of salt(s), or the corresponding acid(s).

Embodiment 22. The enzyme formulation of any of the preceding embodiments, which comprises less than 2% w/w of salt(s), or the corresponding acid(s).

Embodiment 23. The enzyme formulation of any of the preceding embodiments, which further comprises a protease inhibitor.

Embodiment 24. The enzyme formulation of any of the preceding embodiments, which further comprises a boronic acid protease inhibitor or a derivative thereof.

Embodiment 25. The enzyme formulation of any of the preceding embodiments, which further comprises a phenylboronic acid protease inhibitor or a derivative thereof.

Embodiment 26. The enzyme formulation of any of the preceding embodiments, which further comprises a formyl phenylboronic acid protease inhibitor, such as 4-FPBA.

Embodiment 27. The enzyme formulation of any of embodiments 1-23, which further comprises a peptide aldehyde protease inhibitor.

Embodiment 28. The enzyme formulation of the preceding embodiment, wherein the peptide aldehyde has the formula P-B2-B1-B0-H, wherein
B0 is an amino acid selected from the group consisting of leucine (Leu), methionine (Met), phenylalanine (Phe), tyrosine (Tyr), or valine (Val);
B1 is an amino acid selected from the group consisting of alanine (Ala), glycine (Gly), or valine (Val);
B2 is an amino acid selected from the group consisting of arginine (Arg), glycine (Gly), leucine (Leu), phenylalanine (Phe), or valine (Val);
P is a N-terminal protection group, preferably benzyloxycarbonyl or methoxycarbonyl; and
H is hydrogen.

Embodiment 29. The enzyme formulation of the preceding embodiment, wherein the peptide aldehyde has the formula Cbz-Gly-Ala-Tyr-H or Cbz-Val-Ala-Leu-H.

Embodiment 30. The enzyme formulation of any of the preceding embodiments, wherein the protease has at least 80% amino acid sequence identity to SEQ ID NO: 1, SEQ ID NO: 2, SEQ ID NO: 3, SEQ ID NO: 4, or SEQ ID NO: 5.

Embodiment 31. The enzyme formulation of any of the preceding embodiments, wherein the protease has at least 85% amino acid sequence identity to SEQ ID NO: 1, SEQ ID NO: 2, SEQ ID NO: 3, SEQ ID NO: 4, or SEQ ID NO: 5.

Embodiment 32. The enzyme formulation of any of the preceding embodiments, wherein the protease has at least 90% amino acid sequence identity to SEQ ID NO: 1, SEQ ID NO: 2, SEQ ID NO: 3, SEQ ID NO: 4, or SEQ ID NO: 5.

Embodiment 33. The enzyme formulation of any of the preceding embodiments, wherein the protease has at least 95% amino acid sequence identity to SEQ ID NO: 1, SEQ ID NO: 2, SEQ ID NO: 3, SEQ ID NO: 4, or SEQ ID NO: 5.

Embodiment 34. The enzyme formulation of any of the preceding embodiments, wherein the protease has at least 96% amino acid sequence identity to SEQ ID NO: 1, SEQ ID NO: 2, SEQ ID NO: 3, SEQ ID NO: 4, or SEQ ID NO: 5.

Embodiment 35. The enzyme formulation of any of the preceding embodiments, wherein the protease has at least 97% amino acid sequence identity to SEQ ID NO: 1, SEQ ID NO: 2, SEQ ID NO: 3, SEQ ID NO: 4, or SEQ ID NO: 5.

Embodiment 36. The enzyme formulation of any of the preceding embodiments, wherein the protease has at least 98% amino acid sequence identity to SEQ ID NO: 1, SEQ ID NO: 2, SEQ ID NO: 3, SEQ ID NO: 4, or SEQ ID NO: 5.

Embodiment 37. The enzyme formulation of any of the preceding embodiments, wherein the protease has at least 99% amino acid sequence identity to SEQ ID NO: 1, SEQ ID NO: 2, SEQ ID NO: 3, SEQ ID NO: 4, or SEQ ID NO: 5.

Embodiment 38. The enzyme formulation of any of embodiments 30-37, wherein the amino acid changes are substitutions.

Embodiment 39. The enzyme formulation of any of the preceding embodiments, which is essentially free of other (non-protease) enzyme(s).

Embodiment 40. The enzyme formulation of any of the preceding embodiments, which is free of preservation agents.

Embodiment 41. The enzyme formulation of any of the preceding embodiments, which is free of benzoates, sorbates, sulfites, phenoxyethanol, and isothiozolinones.

Embodiment 42. The enzyme formulation of any of the preceding embodiments, where there is essentially no solid phase after centrifugation of the composition at 1200 G for 10 minutes.

Embodiment 43. The enzyme formulation of any of the preceding embodiments, which has a turbidity of less than 100 NTU.

Embodiment 44. The enzyme formulation of any of the preceding embodiments, which has a turbidity of less than 50 NTU.

Embodiment 45. The enzyme formulation of any of the preceding embodiments, which has a turbidity of less than 20 NTU.

Embodiment 46. A method for preparing the liquid enzyme formulation of any of the preceding embodiments, comprising
(a) providing a fermentation broth comprising a protease in an amount of at least 1% w/w;
(b) subjecting the fermentation broth to a flocculation and a membrane filtration to provide a concentrated protease solution; and
(c) mixing the protease solution with glycerol.

Embodiment 47. The method of the preceding embodiment, which further comprises:
(d) evaporating water at a partial vacuum to increase the protease concentration.

Embodiment 48. The method of embodiment 46 or 47, wherein the membrane filtration comprises an ultra-filtration.

Embodiment 49. A method for preparing the liquid enzyme formulation of any of the preceding embodiments, comprising
(a) spray-drying or freeze-drying a protease solution to provide a powder comprising the protease; and
(b) mixing the powder with water and glycerol.

Embodiment 50. A method for preparing a liquid detergent composition, comprising mixing the liquid enzyme formulation of any of the preceding embodiments with a surfactant and a detergent builder; preferably the detergent builder is a non-phosphorus builder/chelator.

Embodiment 51. The method of the preceding embodiment, wherein the final concentration of the surfactant is 0.5-40% w/w, preferably 0.5-25% w/w.

Embodiment 52. The method of the preceding embodiment, wherein the final concentration of the detergent builder is 0.5-40% w/w, preferably 0.5-25% w/w.

Embodiment 53. The method of the preceding embodiment, wherein the final concentration of the protease is 0.0001-1% w/w active enzyme protein, preferably 0.0005-0.5% w/w active enzyme protein.

### EXAMPLES

Chemicals were commercial products of at least reagent grade.

### Example 1

High Strength Liquid Protease Formulations with Glycerol

The formulations below (A-D) were all made by mixing a flocculated and concentrated (by ultra-filtration) liquid protease solution with glycerol, and subsequent evaporation to >10.4% active enzyme protein (AEP), and with/without subsequent further addition of water, glycerol and/or disubstituted alaninamide. The pH of the compositions was adjusted as needed using acetic acid or sodium hydroxide.

### Composition A

10.4% w/w active enzyme protein (Liquanase from Novozymes)
60% w/w glycerol
0.325% w/w disubstituted alaninamide (peptide aldehyde protease inhibitor)
pH 5.5

### Composition B

11.6% w/w active enzyme protein (Liquanase from Novozymes)
50% w/w glycerol
0.36% w/w disubstituted alaninamide (peptide aldehyde protease inhibitor)
pH 5.5

### Composition C

12.3% w/w active enzyme protein (Coronase from Novozymes)
50% w/w glycerol
pH 5.5

### Composition D

19.1% w/w active enzyme protein (Coronase from Novozymes)
47.3% w/w glycerol
pH 5.5

### Results

Enzyme stability was measured as residual activity relative to a reference stored at −18° C. Physical stability was evaluated by visual inspection. “Clear” corresponds to NTU<100.

TABLE 1
Stability of compositions A-D.

Composition

| Property | A | B | C | D |
|---|---|---|---|---|
| Enzyme stability | — | 100% | 100% | 97% |
| (4 weeks at 25° C.) | | | | |
| Enzyme stability | — | — | 95% | 93% |
| (13 weeks at 25° C.) | | | | |
| Physical stability | Clear | Clear | Clear | Clear |
| (4 weeks at 25° C.) | | | | |
| Physical stability | Clear | — | Clear | Clear |
| (13 weeks at 25° C.) | | | | |

As shown in Table 1, compositions A-D exhibit excellent stability after storage for at least 13 weeks at 25° C.

### Example 2

High Strength Liquid Protease Formulations with Propylene Glycol

The compositions below (E and F) were made using the same procedure as in Example 1. The compositions were stored for 2 and 4 weeks at 40° C. before evaluation.

### Composition E

12% w/w active enzyme protein (Liquanase from Novozymes)
60% w/w glycerol
0.38% w/w disubstituted alaninamide (peptide aldehyde protease inhibitor)
pH 5.5

### Composition F

12% w/w active enzyme protein (Liquanase from Novozymes)
40% w/w glycerol
20% w/w propylene glycol (1,2-propanediol)
0.38% w/w disubstituted alaninamide (peptide aldehyde protease inhibitor)
pH 5.5

TABLE 2
Visual evaluation of physical stability of compositions E and F.

Composition

| Property | E | F |
|---|---|---|
| Physical stability | Clear | Precipitated |
| (2 weeks at 40° C.) | | |
| Physical stability | Clear | Precipitated |
| (4 weeks at 40° C.) | | |

‘Clear’ corresponds to NTU < 100 with no visible precipitation.
‘Precipitated’ denotes clearly visible precipitation.

The data in Table 2 show that composition E, which contains only glycerol, has much better physical stability than composition F, which contains a large amount (~33%) of propylene glycol.

Compositions E and F were also made with 10.1%, 10.7%, and 11.4% w/w active enzyme protein, and the physical stability data were identical to those presented in Table 2.

1-16. (canceled)

17. A liquid enzyme formulation, comprising
(a) 10-30% w/w active enzyme protein of protease,
(b) at least 40% w/w of glycerol, and
(c) less than 10% w/w of salt(s) of formate, acetate, citrate, chloride, or the corresponding acid(s);
wherein pH of the formulation is in the range of 4-7.

18. The enzyme formulation of claim 17, wherein the protease is a serine protease.

19. The enzyme formulation of claim 18, wherein the protease is a subtilisin.

20. The enzyme formulation of claim 17, which comprises less than 10% w/w of monopropylene glycol.

21. The enzyme formulation of claim 17, which comprises at least 45% w/w of glycerol.

22. The enzyme formulation of claim 17, which comprises less than 10% w/w of salt(s) or the corresponding acid(s).

23. The enzyme formulation of claim 17, which further comprises a protease inhibitor.

24. The enzyme formulation of claim 23, wherein the protease inhibitor is a boronic acid or a peptide aldehyde.

25. The enzyme formulation of claim 23, wherein the protease inhibitor is 4-formyl-phenyl-boronic acid.

26. The enzyme formulation of claim 23, wherein the protease inhibitor is a peptide aldehyde with the formula P-B2-B1-B0-H, wherein
B0 is an amino acid selected from the group consisting of leucine (Leu), methionine (Met), phenylalanine (Phe), tyrosine (Tyr), or valine (Val);
B1 is an amino acid selected from the group consisting of alanine (Ala), glycine (Gly), or valine (Val);
B2 is an amino acid selected from the group consisting of arginine (Arg), glycine (Gly), leucine (Leu), phenylalanine (Phe), or valine (Val);
P is a N-terminal protection group, preferably benzyloxycarbonyl or methoxycarbonyl; and
H is hydrogen.

27. The enzyme formulation of claim 26, wherein the peptide aldehyde has the formula Cbz-Gly-Ala-Tyr-H or Cbz-Val-Ala-Leu-H.

28. The enzyme formulation of claim 17, wherein the protease has at least 90% amino acid sequence identity to SEQ ID NO: 1, SEQ ID NO: 2, SEQ ID NO: 3, SEQ ID NO: 4, or SEQ ID NO: 5.

29. The enzyme formulation of claim 17, which comprises less than 1% w/w of other (non-protease) enzyme(s).

30. The enzyme formulation of claim 17, wherein is essentially free of other enzyme(s).

31. The enzyme formulation of claim 17, which is free of preservation agents.

32. The enzyme formulation of claim 17, which is free of benzoates, sorbates, sulfites, phenoxyethanol, and isothiozolinones.

33. The enzyme formulation of claim 17, wherein there is essentially no solid phase after centrifugation of the composition at 1200 G for 10 minutes, or which has a turbidity of less than 100 NTU.

34. A method for preparing the liquid enzyme formulation of claim 17, said method comprising
(a) providing a fermentation broth comprising a protease in an amount of at least 1% w/W;
(b) subjecting the fermentation broth to a flocculation and a membrane filtration to provide a concentrated protease solution;
(c) mixing the protease solution with glycerol; and
(d) evaporating water at a partial vacuum to increase the protease concentration.

35. A method for preparing the liquid enzyme formulation of claim 17, said method comprising
(a) spray-drying a protease solution to provide a powder comprising the protease; and
(b) mixing the powder with water and glycerol.

36. A method for preparing a liquid detergent composition, said method comprising mixing the liquid enzyme formulation of claim 17 with a surfactant and a detergent builder.
